(19) United States
(12) Patent Application Publication
(10) Pub. No.: US 2019/0133573 A1
(43) Pub. Date: May 9, 2019

(54) SUTURING KIT

(71) Applicant: Dura Tap LLC, Wayne, PA (US)

(72) Inventors: David Greg Anderson, Moorestown, NJ (US); Mark F. Kurd, Bryn Mawr, PA (US); Jay Tapper, Wayne, PA (US); Jens Johnson, Austin, TX (US)

(21) Appl. No.: 15/802,978

(22) Filed: Nov. 3, 2017

(51) Int. Cl. A61B 17/04; A61B 17/062; A61B 17/00

(52) U.S. Cl. CPC A61B 17/0482; A61B 17/062; A61B 17/06061; A61B 17/00491; A61B 17/0469

(57) ABSTRACT

A suturing kit includes a needle, a suture, a suturing device, a surgical patch and a sealed package. The suture attaches to the needle. The suturing device includes a portion configured to be inserted into a patient during a surgical procedure, a needle holder configured to receive the needle in a retracted position and at least one actuator configured to drive the needle in an advance direction toward a released condition. The surgical patch includes an opening large enough to receive either at least a portion of the needle holder adjacent a distal tip of the needle holder or the needle. The sealed package contains the needle, the suture, the suturing device and the surgical patch.

### BACKGROUND

The present disclosure relates generally to surgery and the placement of sutures, and more particularly to devices and methods for suture repair of the tissue.

Surgical closure using sutures is one approach to tissue repair. Oftentimes a needle driver or similar device is used to locate and to pass a suture needle through the tissue to be repaired. The suture needle attaches at one end to a predetermined length of suture, which can be stored in a suture package. Other wound closure devices, such as staples, and other repair devices, like mesh or patch reinforcements, are frequently used for repair.

Locating the tissue repair devices, especially when working in areas where it is difficult for a surgeon to access such as when working through a tubular retractor or other portal, can be very challenging.

### SUMMARY

In view of the foregoing, a suturing kit includes a needle, a suture, a suturing device, a surgical patch and a sealed package. The suture attaches to the needle. The suturing device includes a portion configured to be inserted into a patient during a surgical procedure, a needle holder configured to receive the needle in a retracted position and at least one actuator configured to drive the needle in an advance direction toward a released condition. The surgical patch includes an opening large enough to receive either at least a portion of the needle holder adjacent a distal tip of the needle holder or the needle. The sealed package contains the needle, the suture, the suturing device and the surgical patch.

Another example of a suturing kit includes a surgical patch, a plurality of sutures, a plurality of needles, at least one suturing device and a sealed package. The plurality of sutures connect with the surgical patch. Each needle connects with a respective suture. The at least one suturing device includes a portion configured to be inserted into a patient during a surgical procedure, and a needle holder configured to receive or hold a respective needle of the plurality of needles. The sealed package contains the plurality of sutures, the plurality of needles, the at least one suturing device and the surgical patch.

### BRIEF DESCRIPTION OF THE DRAWINGS

FIG. 1 is a perspective view of a suturing kit including suturing devices, sutures, a suture holder, a knot pusher, and a surgical patch.

FIG. 2 is a schematic depiction of the suture holder connected with a vacuum source prior to the sutures being received in the suture holder.

FIG. 3 is a perspective view of an alternative suturing device for use with surgical patches.

FIG. 4 is a perspective view of the surgical patch disclosed in FIG. 1.

FIG. 5 is a perspective view of an alternative surgical patch.

FIG. 6 is a perspective view of another alternative surgical patch.

FIG. 7 is a perspective view of an alternative suturing kit including another alternative surgical patch.

### DETAILED DESCRIPTION

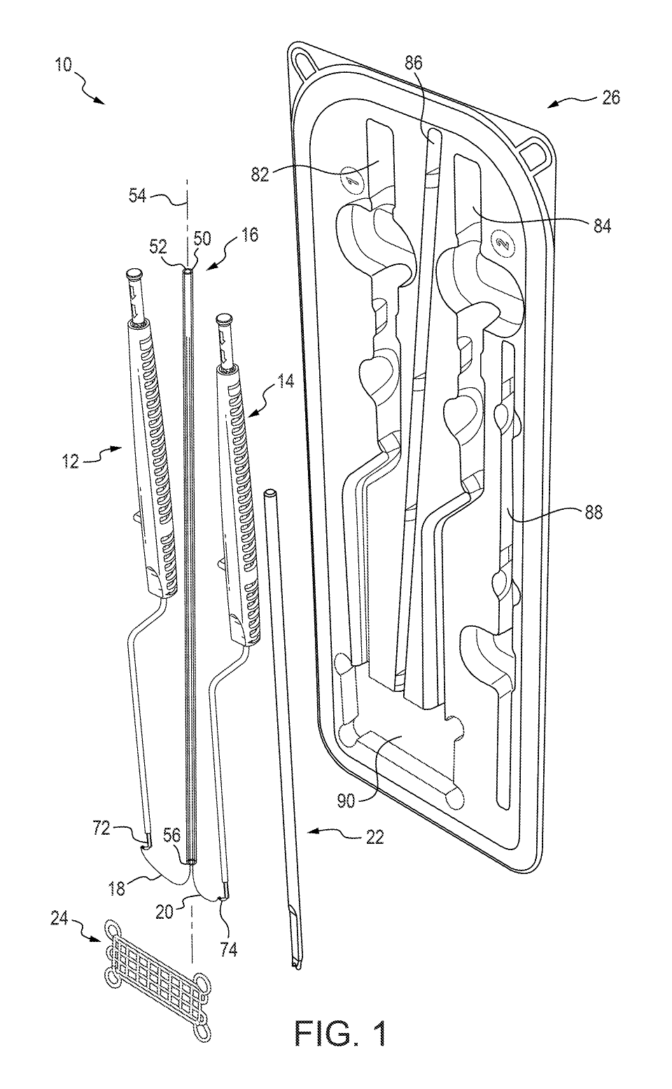

With reference to FIG. 1, a suturing kit 10 includes at least one suturing device (two suturing devices 12, 14 are shown in FIG. 1), a suture holder 16, at least one suture (two sutures 18, 20 are shown in FIG. 1), a knot pusher 22 and a surgical patch 24. The suturing devices 12, 14, the suture holder 16, the sutures 18, 20 the knot pusher 22 and the surgical patch 24 are received in a package including a package base 26 and are covered by a cover sheet (not shown), similar to a known cover sheet for packaging sterile surgical instruments, to provide a sealed package.

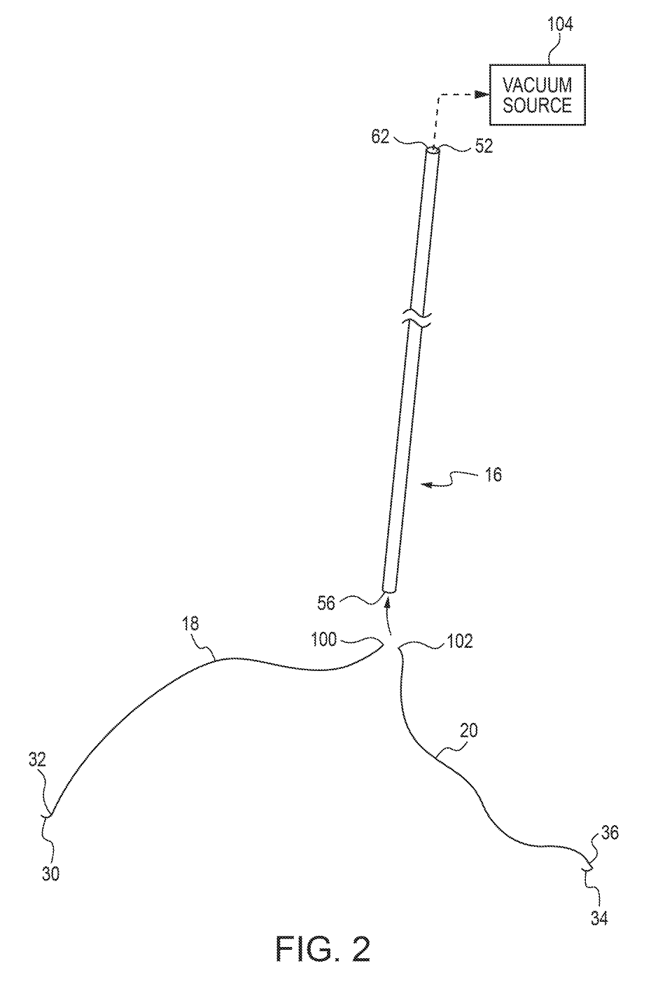

With reference to FIG. 2, the first suture 18 has a first needle 30 attached at one end 32. The second suture 20 has a second needle 34 attached at one end 36. The needles 30, 34 can be similar to commercially available curved needles made from known materials. The needles 30, 34 could also be formed from a malleable, or flexible, material such that the needles 30, 34 could follow a curve when positioned within a curved needle holder, and then later straighten after exiting the needle holder. The needles 30, 34 can take other configurations, such as straight. Also, the needles 30, 34 could be formed as part of the sutures 18, 20 e.g., the needles 30, 34 could be a rigid end of each suture 18, 20 that is configured so as to be suitable to pass through animal tissue.

The sutures 18, 20 depicted in FIGS. 1 and 2 are single-armed sutures each having a respective needle 30, 34 at one end; however, a double-armed suture that is looped over on itself could be stored in the suture holder 16. The suture holder 16 is shown as transparent so that the sutures 18, 20 received within the suture holder 16 are visible. In contrast to known suture packages, the sutures 18, 20 within the suture holder 16 are not each wound around itself several times.

The suture holder 16 includes an elongate wall 50 defining a suture receiving passage 52 that is elongated along a longitudinal axis 54 and terminates at a suture exit opening 56. The elongate wall 50 has a closed cross section normal to the longitudinal axis 54 in the illustrated embodiment. The elongate wall 50 is circular in cross section normal to the longitudinal axis 54; however, the elongate wall 50 can take other shapes and need not be a closed cross section. For example, the elongate wall 50 may be C-shaped or U-shaped in cross section. The suture holder 16 has a smooth interior surface so as to inhibit the sutures 18, 20 from being caught or snagged during deployment from the suture holder 16. The suture receiving passage 52 also terminates at a second opening 62 spaced from the suture exit opening 56 along the longitudinal axis 54. Accordingly, the suture receiving passage 52 extends entirely through the suture holder 16 along the longitudinal axis 54 and has a configuration similar to a conventional drinking straw. The suture receiving passage 52 has a width, which is measured in the plane normal to the longitudinal axis 54 and in the illustrated embodiment is a diameter of the circular cross section, that is at least twice the diameter of one of the sutures 18, 20, which allows the suture receiving passage 52 to receive the sutures 18, 20 side by side. The suture receiving passage 52 is longest along the longitudinal axis 54, which is several times greater than the width of the suture receiving passage 52.

As illustrated, the portion of the first suture 18 from adjacent to the second opening 62 to the suture exit opening 56 is aligned parallel to the longitudinal axis 54. Similarly, the portion of the second suture 20 from adjacent to the second opening 62 to the suture exit opening 56 is aligned parallel to the longitudinal axis 54. Both the first suture 18 and the second suture 20 may not be exactly straight, especially since the sutures 18, 20 are each a thread-like material, so “parallel” includes not only the case where the angle made by either the first suture 18 or the second suture 20 and the longitudinal axis 54 is 0°, but also the case where the angle is −5 to +5°. Accordingly, each of the first suture 18 and the second suture 20 from adjacent to the second opening 62 to the suture exit opening 56 within the suture receiving passage 52 can be referred to as aligned along the longitudinal axis 54.

The suturing kit 10 shown in FIG. 1 includes the suturing devices 12 and 14, which are each configured to be inserted into a patient during a surgical procedure. The suturing devices 12 and 14 depicted in FIG. 1 are more particularly described in U.S. patent application Ser. No. 15/646,521 filed Jul. 11, 2017. Accordingly, the details of the suturing devices 12, 14 have been omitted for the sake of brevity. The first suturing device 12 includes a first needle holder 72, which holds the first needle 30 (FIG. 2). The second suturing device 14 includes a second needle holder 74 that stores the second needle 34 (FIG. 2). Other types of suturing devices, such as conventional needle drivers, could be provided with the suturing kit 10 in addition to or in lieu of the depicted suturing devices 12 and 14.

With a reference to FIG. 1, the package base 26 includes a first suturing device recess 82 that receives the first suturing device 12, a second suturing device recess 84 that receives the second suturing device 14, and a suture holder recess 86 that receives the suture holder 16. The suture holder recess 86 is positioned between the first suturing device recess 82 and the second suturing device recess 84. The package base 26 also includes a knot pusher recess 88 that receives the knot pusher 22 and a patch recess 90 that receives the surgical patch 24. A section of the first suture 18 residing outside of the suture holder 16 extends away from a section of the second suture 20 residing outside of the suture holder 16 when the first suturing device 12 is received in the first suturing device recess 82, the second suturing device 14 is received in the second suturing device recess 84 and the suture holder 16 is received in the suture holder recess 86. The section of the first suture 18 residing outside of the suture holder 16 and the section of the second suture 20 residing outside of the suture holder 16 resides in the patch recess 90 when the first suturing device 12 is received in the first suturing device recess 82, the second suturing device 14 is received in the second suturing device recess 84 and the suture holder 16 is received in the suture holder recess 86.

The knot pusher 22 depicted in FIGS. 1 and 2 can be similar to known knot pushers.

A method of assembling the suturing kit 10 includes inserting a free end 100 of the first suture 18 and a free end 102 of the second suture 20 through the suture exit opening 56 into the suture receiving passage 52 of the suture holder 16. When assembling the suturing kit 10, a vacuum source 104 can be connected with the suture holder 16 to cover the second opening 62 and draw air through the suture receiving passage 52 to draw the sutures 18, 20 through the suture exit opening 56 into the suture receiving passage 52.

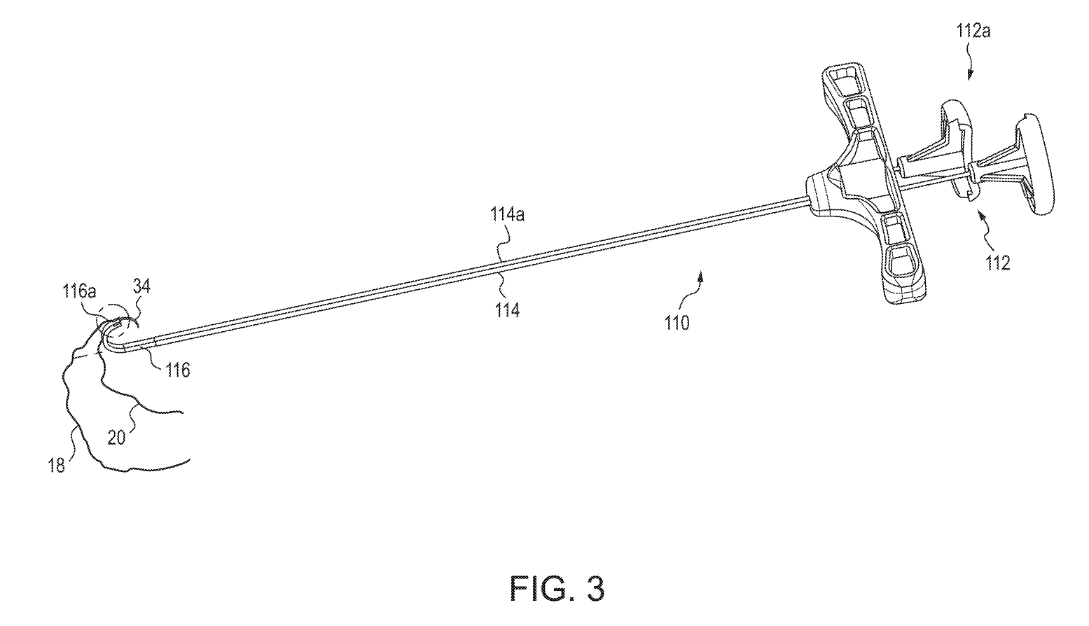

With reference to FIG. 3, an alternative suturing device 110 that can be used with the surgical patch 24 in FIG. 1 is shown. The alternative suturing device 110 is more particularly described in PCT patent application no. PCT/US2016/63276 filed Nov. 22, 2016 and will not be fully described for the sake of brevity. The alternative suturing device 110 includes a first actuator 112, a second actuator 112a, a first elongate body 114, a second elongate body 114a, a first needle holder 116 and a second needle holder 116a.

Actuation of the first actuator 112 moves the first needle 30 (not visible in FIG. 3, see FIG. 2) from a retracted position and drives the first needle 30 in an advance direction toward a released condition. Similarly, actuation of the second actuator 112a moves the second needle 34 from a retracted position and drives the second needle 34 in an advance direction toward a released condition.

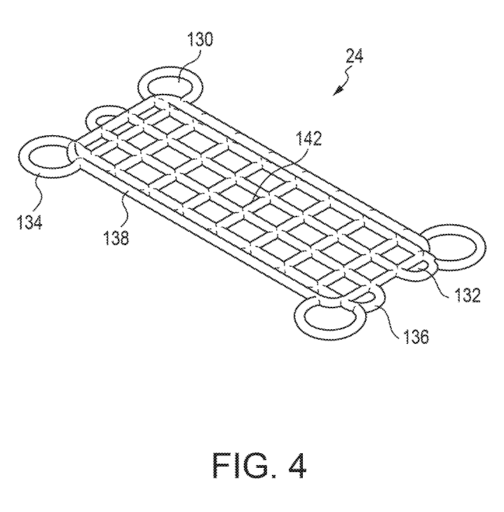

With reference to FIG. 4, the surgical patch 24 is shown in more detail. The surgical patch 24 is depicted in FIG. 4 as a mesh that can be made from woven or interconnected threads of animal tissue, polymer, or fabric. The surgical patch 24 includes an opening (a plurality of openings 130, 132, are shown for the surgical patch 24 depicted in FIG. 4) large enough to receive at least a portion of the needle holder, e.g., needle holders 72, 74, 116, and 116a adjacent a distal tip of the needle holder. In other words, the distal tip of the needle holder 72, 74, 116, 116a can pass through either corner openings 130 or lateral openings 132 prior to deploying the needle 30, 34 from the respective needle holder during a tissue repair procedure. The openings 130, 132 can also be large enough to receive either needle 30 or needle 34. As explained in more detail in PCT patent application number PCT/US 2016/63276 and U.S. patent application Ser. No. 15/646,521, a physician using either the suturing devices 12, 14 or the suturing device 110, can position the distal-most tip under an internal side of the target tissue to be repaired. The physician can pull up on the suturing device such that the distal-most tip “tents” the target tissue to be repaired providing a visual indication of where the needle 30, 34 is to be delivered. The “tented” location can be aligned with one of the corner openings 130 or one of the lateral openings 132 in the surgical patch 24, and then the actuator on the suturing device 12, 14 or 110 can be actuated driving the needle in the advance direction toward the released condition so that the needle will travel through the respective opening 130 or 132. Alternatively, the needle 30, 34 could be advanced through the tissue that is to be repaired and then the surgical patch 24 can be placed over top the needle 30, 34, which may or may not advance entirely through the target tissue to be repaired. The suture 18, 20 can then be used to fix the surgical patch 24 to the target tissue to be repaired. This procedure can be duplicated for other openings on the surgical patch 24 to affix the surgical patch 24 to the target tissue to be repaired.

The surgical patch 24 depicted in FIG. 4 includes at least one eyelet, e.g., corner eyelets 134 and lateral eyelets 136. In the illustrated embodiment, the eyelets 134, 136 defined the respective openings 130, 132. The eyelets 134, 136 are positioned closer to a peripheral edge 138 of the surgical patch 24 as compared to the center 142 of the surgical patch. Each of the eyelets 134, 136 extends outwardly from the peripheral edge 138 of the surgical patch 24. In the illustrated embodiment, the surgical patch 24 takes a generally rectangular configuration in plan view. The surgical patch, however, can take other shapes and configurations.

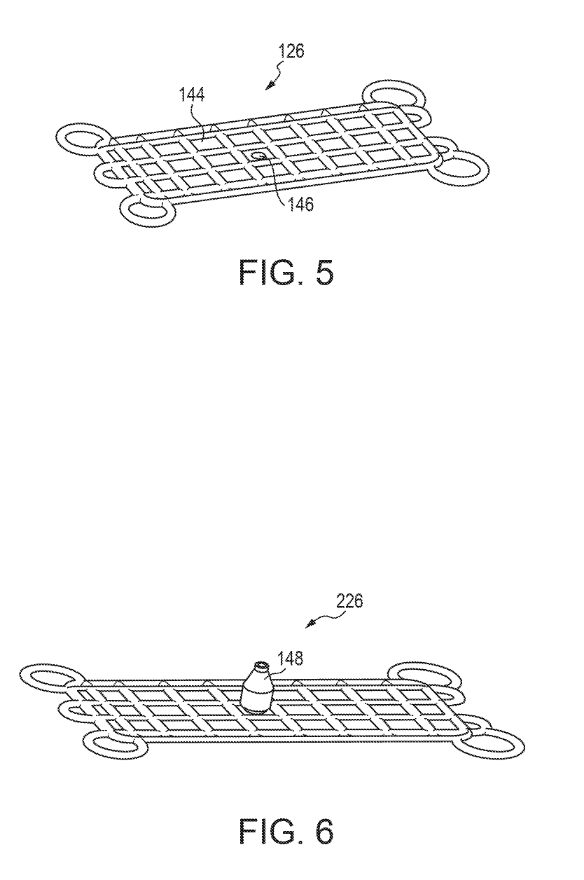

FIGS. 5 and 6 depict alternative surgical patches 126, 226, respectively. Surgical patch 126 in FIG. 5 is similar in all respects to the surgical patch 24 depicted in FIG. 4 with the exception that the surgical patch 126 also includes a membrane 144, which can be a fluid-tight membrane or an amniotic membrane, for example. Where the membrane 144 is a fluid-tight membrane it is configured to prohibit the passage of fluid therethrough. Such a fluid-tight membrane 144 can be particularly useful when the surgical patch 126 is to patch an area of the body where a fluid-tight repair is desired. The fluid-tight membrane 144 can include an aperture 146 extending therethrough. The aperture 146 is positioned in the center of the surgical patch 126 in the embodiment illustrated in FIG. 5; however, the aperture 146 can be located elsewhere or no aperture need be provided. Additionally, a plurality of apertures 146 may be provided. The apertures 146 can be useful to allow for the introduction of fluid and other materials into the area around the tissue site that is to be repaired. Adhesives or other biocompatible products can be used to cover and plug the aperture 146 during the tissue repair procedure.

The surgical patch 226 depicted in FIG. 6 is similar in all respects to the surgical patch 126 depicted in FIG. 5 with the exception of the addition of a one-way valve 148 associated with the aperture 146. The one-way valve 148 can allow entry of fluid into the site of the body with the tissue to be repaired, for example to allow for the injection of drugs. The one-way valve 148, however, would preclude flow of fluid in the opposite direction.

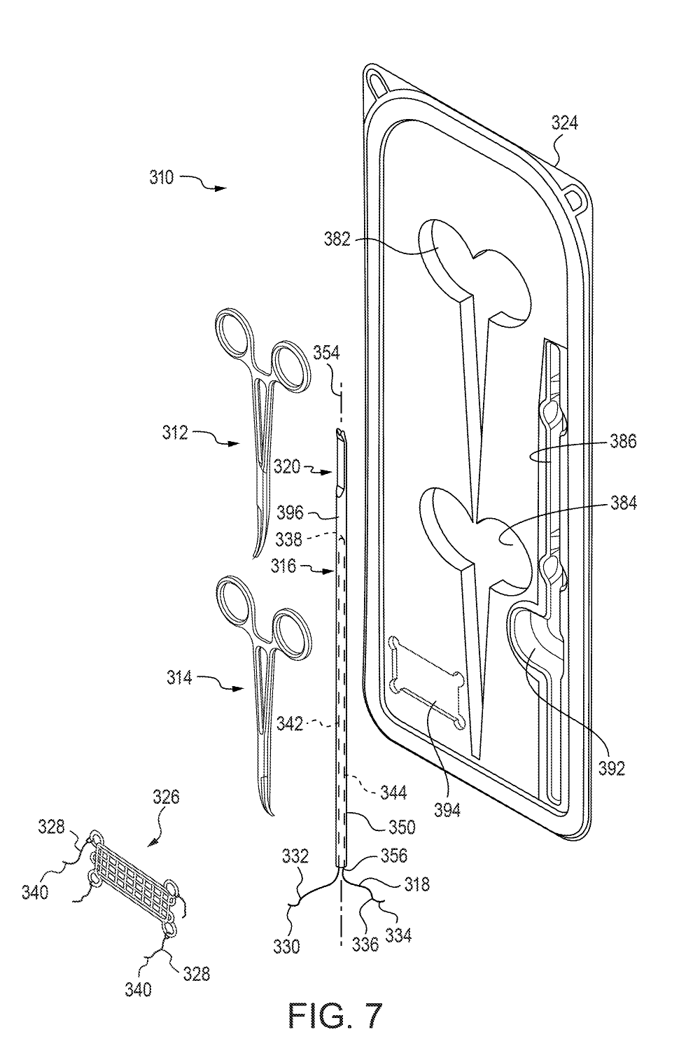

FIG. 7 depicts an alternative surgical patch 326 that is similar the surgical patch 24 depicted in FIG. 4. The surgical patch 326 depicted in FIG. 7, however, includes a plurality of sutures 328 each connected with the surgical patch 326. A plurality of needles 340 is also provided where each needle 340 connects with a respective suture 328.

With reference to FIG. 7, an alternative suturing kit 310 includes at least one suturing device (two suturing devices 312, 314 are shown), a suture holder 316, a suture 318, and a knot pusher 320. The suturing devices 312, 314, the suture holder 316, the suture 318, and the knot pusher 320 are received in a package including a package base 324 and are covered by a cover sheet (not shown) similar to a known cover sheet for packaging sterile surgical instruments.

The suturing devices 312 and 314 are each configured to be inserted into a patient during a surgical procedure. The suturing devices 312 and 314 depicted in FIG. 7 are similar to needle drivers shaped similar to a hemostat and include a respective needle holder at a distal end portion thereof. Other types of suturing devices, such as other conventional needle drivers, could be provided with the suturing kit 310 in addition to or in lieu of the depicted suturing devices 312 and 314.

The suture 318 has a first needle 330 attached at a first end 332 and a second needle 334 attached at a second, opposite, end 336. The needles 330, 334 can be similar the needles 30, 34 described above. The suture 318 depicted in FIG. 7 is a double-armed suture having a respective needle 330, 334 at each end; however, the suture holder 316 can also be used with single-armed sutures.

The suture 318 (shown in phantom inside the suture holder 316) is doubled over within the suture holder 316 to provide a half loop section 338. A first section 342 of the suture 318 extends from the half loop section 338 toward the first end 332. A second section 344 of the suture 318 extends from the half loop section 338 toward the second end 336 of the suture 318. In contrast to known suture packages, the suture 318 within the suture holder 316 is not wound around itself several times.

The suture holder 316 includes an elongate wall 350 defining a suture receiving passage (similar to the suture receiving passage 52 described above) that is elongated along a longitudinal axis 354 and terminates at a suture exit opening 356. The elongate wall 350 has a closed cross section normal to the longitudinal axis 354 in the illustrated embodiment. The elongate wall 350 is circular in cross section normal to the longitudinal axis 354; however, the elongate wall 350 can take other shapes and need not be a closed cross section. The elongate wall 350 also includes an interior surface, which can be smooth so as to inhibit the suture 318 from being caught or snagged during its deployment from the suture holder 316. The elongate wall 350 also includes an exterior surface, which can also be smooth and parallel or coaxial with the interior surface.

The suture receiving passage also terminates at a second opening, which is covered by the knot pusher 320 in FIG. 7, spaced from the suture exit opening 356 along the longitudinal axis 354. The suture receiving passage has a width, which is measured in the plane normal to the longitudinal axis 354 and in the illustrated embodiment is a diameter of the circular cross section, that is at least twice the diameter of the suture 318, which allows the suture receiving passage to receive the suture 318 such that the suture is folded over forming the half loop section 338. The suture receiving passage is longest along the longitudinal axis 354, which is several times greater than the width of the suture receiving passage.

When the suture 318 is received in the suture receiving passage, the suture 318 is folded over forming the half loop section 338, which is spaced from the suture exit opening 356. As illustrated, the first section 342 of the suture 318 from adjacent to the half loop section 338 to the suture exit opening 356 within the suture receiving passage is aligned parallel to the longitudinal axis 354. Similarly, the second section 344 of the suture 318 from adjacent to the half loop section 338 to the suture exit opening 356 within the suture receiving passage is aligned parallel to the longitudinal axis 354. Both the first section 342 and the second section 344 may not be exactly straight, especially since the suture 318 is a thread-like material, so “parallel” includes not only the case where the angle made by either the first section 342 or the second section 344 and the longitudinal axis 354 is 0°, but also the case where the angle is −5 to +5°. Accordingly, each of the first section 342 and the second section 344 of the suture 318 from adjacent to the half loop section 338 to the suture exit opening 356 within the suture receiving passage can be referred to as aligned along the longitudinal axis 354. As illustrated, there is only one point along the suture 318 within the suture receiving passage 352 where the suture is doubled over, which is the half loop section 338.

The suturing kit 310 includes the suturing devices 312 and 314, which are each configured to be inserted into a patient during a surgical procedure. The suturing devices 312 and 314 depicted in FIG. 7 are similar to needle drivers shaped similar to a hemostat. The suturing devices 312 and 314 depicted in FIG. 7 each include a needle holder at a distal end thereof configured to receive or hold a respective needle, 330, 334 and 340. Other types of suturing devices, such as other conventional needle drivers, could be provided with the suturing kit 310 in addition to or in lieu of the depicted suturing devices 312 and 314.

The package base 324 includes a first suturing device recess 382 that receives the first suturing device 312, a second suturing device recess 384 that receives the second suturing device 314, and a suture holder recess 386 that receives the suture holder 316. The package base 324 can be provided with a gripping cutout 392 that extends outwardly from the suture holder recess 386. The gripping cutout 392 allows for the insertion of fingers to facilitate removal of the suture holder 316 and the knot pusher 320. The package base 324 can be provided with a surgical patch recess 394 that receives the surgical patch 326.

The knot pusher 320 depicted in FIG. 7 connects with the suture holder 316. As illustrated, the knot pusher 320 connects with an end 396 of the suture holder 316 opposite from the suture exit opening 356. The knot pusher 320 can snap into the opening provided at the end 396 after the suture 318 has been sucked into the suture passage, for example through the use of the vacuum source 104, which is shown in FIG. 2.

Suturing kits and a method of assembling a suturing kit have been described above with particularity. Modifications and alterations will occur to those upon reading and understanding the above detailed description. The invention, however, is not limited to only the embodiments described above. Instead, the invention is broadly defined by the appended claims and the equivalents thereof. Also, as used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

1. A suturing kit comprising:
a needle;
a suture attached to the needle;
a suturing device having a portion configured to be inserted into a patient during a surgical procedure, the suturing device including a needle holder configured to receive the needle in a retracted position and at least one actuator configured to drive the needle in an advance direction toward a released condition;
a surgical patch including an opening large enough to receive either at least a portion of the needle holder adjacent a distal tip of the needle holder or the needle; and
a sealed package containing the needle, the suture, the suturing device and the surgical patch.

2. The suturing kit of claim 1, wherein the surgical patch includes at least one of animal tissue, a polymer, a mesh, fabric, an amniotic membrane and a fluid-tight membrane.

3. The suturing kit of claim 1, wherein the surgical patch includes at least one eyelet defining the opening.

4. The suturing kit of claim 3, wherein the eyelet is positioned closer to a peripheral edge of the surgical patch as compared to the center of the surgical patch.

5. The suturing kit of claim 3, wherein the eyelet extends outwardly from a peripheral edge of the surgical patch.

6. The suturing kit of claim 1, wherein the surgical patch includes a fluid-tight membrane configured to prohibit passage of fluid therethrough.

7. The suturing kit of claim 6, wherein the fluid-tight membrane includes an aperture.

8. The suturing kit of claim 7, wherein the surgical patch includes a one-way valve associated with the aperture.

9. The suturing kit of claim 1, further comprising a suture holder in the sealed package holding at least a portion of the suture, wherein the suture holder is separate from the at least one suturing device so as not to be inserted into the patient during the surgical procedure.

10. The suturing kit of claim 9, wherein the suture holder includes an elongate wall defining a suture receiving passage that is elongated along a longitudinal axis and terminates at a suture exit opening, and the suture within the suture receiving passage is aligned along the longitudinal axis.

11. The suturing kit of claim 10, wherein the suture receiving passage terminates at a second opening spaced from the suture exit opening along the longitudinal axis.

12. The suturing kit of claim 1, wherein the suturing device includes a second needle holder configured to receive a second needle in a retracted position and the at least one actuator is configured to drive the second needle in an advance direction toward a released condition.

13. The suturing kit of claim 1, wherein the suturing device is a first suturing device and the needle holder is a first needle holder, the suturing kit further comprising a second suturing device, which is a separate instrument from the first suturing device, including a second needle holder.

14. The suturing kit of claim 1, wherein the needle holder includes a distal opening through which the needle exits when moving from the retracted position toward the released condition, wherein the suture extends through distal opening when the needle is in the retracted position.

15. A suturing kit comprising:
a surgical patch;
a plurality of sutures connected with the surgical patch;
a plurality of needles, each needle connected with a respective suture;
at least one suturing device having a portion configured to be inserted into a patient during a surgical procedure and a needle holder configured to receive or hold a respective needle of the plurality of needles; and
a sealed package containing the plurality of sutures, the plurality of needles, the at least one suturing device and the surgical patch.

16. The suturing kit of claim 15, wherein the surgical patch includes at least one of animal tissue, a polymer, a mesh, fabric, an amniotic membrane and a fluid-tight membrane.

17. The suturing kit of claim 15, wherein the surgical patch includes a fluid-tight membrane configured to prohibit passage of fluid therethrough.

18. The suturing kit of claim 17, wherein the fluid-tight membrane includes an aperture.

19. The suturing kit of claim 18, wherein the surgical patch includes a one-way valve associated with the aperture.

20. The suturing kit of claim 15, wherein the needle holder receives each respective needle.
